(19) United States
(12) Patent Application Publication
(10) Pub. No.: US 2010/0057068 A1
(43) Pub. Date: Mar. 4, 2010

(54) GOLD NANOSTRUCTURE AND METHODS OF MAKING AND USING THE SAME

(76) Inventor: KWANGYEOL LEE, Namyangju-si (KR)

Correspondence Address: EDWARDS ANGELL PALMER & DODGE LLP, P.O. BOX 55874, BOSTON, MA, 02205, US

(21) Appl. No.: 12/202,013

(22) Filed: Aug. 29, 2008

(51) Int. Cl. A61B 18/04; C21B 13/00

(52) U.S. Cl. 606/27; 75/300

(57) ABSTRACT

A gold nanostructure, comprising a substrate, a dielectric material, one or more of gold nanoparticles is provided together with related devices and methods.

### BACKGROUND

Materials reduced to the nanoscale can exhibit different physical and chemical properties compared to those on a macroscale. The different properties are due in part to the increased in surface area to volume ratio, which can alter mechanical, electrical, optical and catalytic properties of materials. Such distinctive properties present in the nanosized materials can depend on both the size and shape of the materials.

### SUMMARY

Certain embodiments of the disclosure relate to nanostructures, and methods of making and using the same. Some embodiments relate to gold nanostructures that include, for example, a substrate, a dielectric material coated on the substrate, and one or more gold nanoparticles adhered to the dielectric material-coated substrate. Some embodiments relate to devices that include, for example, one or more of the gold nanostructures disclosed herein, and a radiation energy transmitting conduit connected to a gold nanostructure as described herein.

Some embodiments relate to methods of using the nanostructures disclosed herein, for example, to remove or destroy a cellular or non-cellular tissue. In some aspects, the methods can include, for example, localizing the gold nanostructure to the cellular or non-cellular tissue, and providing radiation to the gold nanostructure to induce heat.

Some embodiments relate to methods of making gold nanostructures disclosed herein. The methods can include, for example, coating a substrate with a dielectric material, and adhering one or more gold nanoparticles to the dielectric material-coated substrate.

Some embodiments relate to methods of making a device, for example, for transmitting heat to a localized area. In some embodiments, the methods can include, for example, connecting one or more gold nanostructures to a radiation energy transmitting conduit.

Some embodiments relate to methods of generating heat at a desired location. The methods can include, for example, localizing a gold nanostructure at a desired location, and providing radiation to the gold nanostructure to induce heat at the desired location.

The foregoing is a summary and thus contains, by necessity, simplifications, generalization, and omissions of detail; consequently, those skilled in the art will appreciate that the summary is illustrative only and is not intended to be in any way limiting. Other aspects, features, and advantages of the devices and/or processes and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE DRAWING

The foregoing and other features of the present disclosure will become more fully apparent from the following description and appended claims, taken in conjunction with the accompanying drawing. Understanding that the drawing depicts only several embodiments in accordance with the disclosure and is, therefore, not to be considered limiting of its scope, the disclosure will be described with additional specificity and detail through use of the accompanying drawing.

FIGS. 1A and 1B illustrate an example of a process of making a gold nanostructure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

Some embodiments of the present disclosure relate generally to gold nanostructures that include, for example, one or more gold nanoparticles, a substrate, and a dielectric material. Also, some embodiments relate to methods of making the gold nanostructures, as well as to methods of using the gold nanostructures to increase the local temperature around the gold. Furthermore, some embodiments relate to devices that include, for example, a gold nanostructure as described herein, a radiation energy transmitting conduit, and a radiation energy source.

Nanoscale gold is known to exhibit surface plasmon resonance (SPR). SPR describes a physical quality acquired by nanosized gold upon exposure to electromagnetic radiation, such as visible (Vis) to near infra red (IR) light. Energy provided from radiation or light can cause oscillation of electrons present in the nanosized gold. When the excited electrons return to their ground state energy level, the extra energy is emitted as heat.

Some embodiments relate to gold nanostructures that can induce heat in localized areas. Various embodiments relate to gold nanostructures that include, for example, a substrate, a dielectric material which partially or completely covers the substrate, and one or more gold nanoparticles that are adhered to the dielectric material-coated substrate. A substrate that is “partially covered” with a dielectric substrate is not completely covered. For example, if a substrate is a thin layer with two planes, “partially covered” can refer to a substrate, for example, in which one of the two planes is covered with a dielectric material. “Partially covered” substrates can also refer to substrates in which only a part of one or both of the planes of the substrate are covered with dielectric material. Furthermore, partially covered can refer to a dielectric material covering from about 30% to about 99% of the substrate or one or more planes of the substrate. In some aspects from about 50% to about 99% of the substrate or one or more planes of the substrate are covered, or from about 65% to about 99% coverage, for example.

As will be described in more detail below, one or more gold nanoparticles can be deposited onto or adhered to at least part of a plane of the substrate where the dielectric material is coated. In some aspects one or both planes of the thin-layered substrate can be coated with the dielectric material, for example, to be completely covered by the dielectric material. In such an example, the gold nanoparticles can adhere either on one side or both sides of the coated substrate.

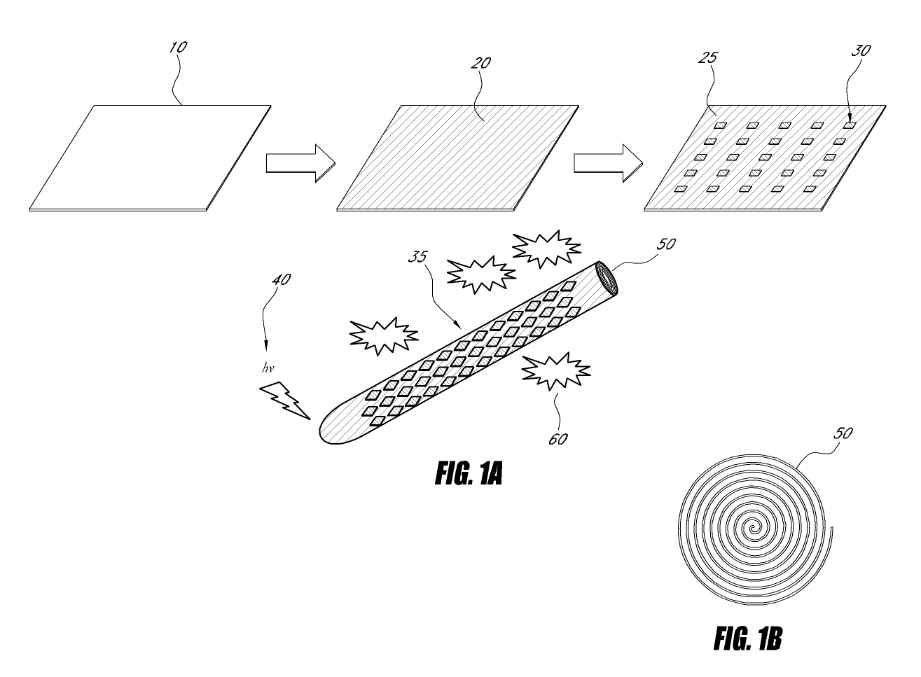

In general, the substrates function as platforms on which one or more gold nanoparticles are deposited or dispersed. Gold nanoparticles can be deposited on the substrate in various patterns or configurations. For example, the gold nanoparticles can be patterned onto the substrate in a regular, semi-regular, or irregular pattern. By way of example, several (e.g., tens, hundreds, thousands) gold nanoparticles can be aligned into several evenly spaced lines. In some embodiments, each individual gold nanoparticle can be arranged such that a certain distance exists between adjacent particles. One illustrative example of manufacturing the gold nanostructure is shown in FIG. 1, which is discussed in more detail below.

In some embodiments, the substrate can be made of stainless steel, titanium, aluminum, or any combination thereof, for example. The substrate can have a variety of shapes including, but not limited to, a layer, a cylinder, a sphere, a rod, a tubular structure, a fiber, any type of hexahedron, and any regular or irregular shaped two-dimensional or three dimensional structures.

In one embodiment, the substrate is stainless steel shaped into a thin layer or a foil. In one embodiment, the stainless steel foil can have two planes and four sides, for example. In some embodiments, the thickness of the foil can be less than the width and/or the length of the planes. In some embodiments, the stainless steel foil layer can further be shaped to be a circle, a triangle, a square, or any kind of polygon, for example.

While the average size of the substrate used in connection with some embodiments herein can vary, in some embodiments, the average size (e.g. diameter, width, length, or height) of the substrate can be between about 10 nanometers (nm) and about 1 meter (m). For example, in some embodiments, the average size (e.g. diameter, width, length, or height) of the substrate used can be in a range from about 10 nm, 10 micrometers (μm), 10 millimeters (mm), 10 centimeters (cm), or 100 cm to about 10 μm, 10 mm, 10 cm, 100 cm, or 1 m. In some embodiments, the average size (e.g. diameter, width, length, or height) of the substrate can be between about 10 nm to about 100 cm. In an illustrative embodiment, the average size (e.g. diameter, width, length, or height) of the substrate in some of such embodiments can be between about 10 nm to about 10 cm, e.g., 10 nm, 20 nm, 30 nm, 40 nm, 50 nm, 60 nm, 70 nm, 80 nm, 90 nm, 1 cm, 2 cm, 3 cm, 4 cm, 5 cm, 6 cm, 7 cm, 8 cm, 9 cm, 10 cm, or more, or any number in between.

In some embodiments, the dielectric material can be a nonconducting material. Also, the dielectric material can be in any form, for example, gas, liquid, gel, semi-solid, and solid. A nonconducting material used in some embodiments herein can include materials that lack movable electric charges and thus lack a low-resistance path for electrical charge flow. Any of a wide variety of non-conducting materials can be used as the dielectric material. Illustrative materials of the dielectric material include, but are not limited to, oxides, such as oxides of iron, aluminum, titanium and other metals. It should be apparent to those having ordinary skill in the art, with the benefit of the instant disclosure that numerous known materials can be used as a dielectric material.

In some embodiments, a metal oxide, such as an oxide of iron, can be used as the dielectric material. In some embodiments, the iron or other elemental metal can be applied to all or part of the substrate. In some embodiments, the substrate can be a material that is more resistant to oxidation than the metal, such as a stainless steel foil, for example. The metal can be coated on all or part of the substrate via a variety of known methods including, but not limited to, a chemical vapor deposition method, an atomic layer deposition method, and the like. These and other methods can be further modified as appropriate by those having ordinary skill in the art in view of the instant disclosure. Once the metal is coated on all or part of the substrate, the metal-coated substrate can be exposed to an atmosphere, such as air or purified oxygen, which is partially or completely composed of oxygen. The metal coated on the surface of the substrate thereby becomes oxidized. For example, in embodiments where iron is coated on a stainless steel foil, the resulting material is a stainless steel foil coated with iron oxide as the dielectric material.

In general, the average thickness of the dielectric material coated on the substrate in certain embodiments can be between about 10 nm to about 500 nm. For example, in some embodiments, the average thickness of the dielectric material coating on the surface of the substrate can be in the range from about 10 nm, 50 nm, 100 nm, 200 nm, 300 nm, or 400 nm to about 50 nm, 100 nm, 200 nm, 300 nm, 400 nm, or 500 nm, or more, or any number in between.

The gold nanoparticles can include various types of gold with various purities, qualities, and or characteristics. In some embodiments, the gold nanoparticles can be made of pure gold. In some other embodiments, the gold nanoparticles can include gold and other substances that may enhance or may not influence the ability of the gold nanoparticle to induce heat by SPR. For example, the nanoparticles can include gold and alloy material(s). In some embodiments, the amount of gold used in one square centimeter area can be between about 0.01 μg to about 5 μg. In some embodiments, the amount of gold used in one square centimeter area can be between about 0.01 μg, 0.1 μg, 0.5 μg, 1 μg, 2 μg, 3 μg, or 4 μg to about 0.1 μg, 0.5 μg, 1 μg, 2 μg, 3 μg, 4 μg, or 5 μg.

The gold nanoparticles disclosed herein can have a variety of shapes, such as, but not limited to, a layer or sheet, a cylinder, a sphere, a rod, a tubular structure, a fiber, any type of hexahedron, or any regular or irregular shaped two-dimensional or three-dimensional structures.

In general, the average size (e.g. diameter, width, length, or height) of the gold nanoparticle can be less than about 2,000 nm. In some embodiments, the average size (e.g. diameter, width, length, or height) of the gold nanoparticle can be between about 1 nm, 10 nm, 100 nm, 500 nm, 800 nm, 1,000 nm, 1,200 nm, 1,500 nm, or 1,800 nm and about 10 nm, 100 nm, 500 nm, 800 nm, 1,000 nm, 1,200 nm, 1,500 nm, 1,800 nm or 2,000 nm, for example. In some embodiments, the average size (e.g. diameter, width, length, or height) of the gold nanoparticle can be between about 10 nm and about 800 nm, e.g. 10 nm, 20 nm, 30 nm, 40 nm, 50 nm, 60 nm, 70 nm, 80 nm, 90 nm, 100 nm, 120 nm, 140 nm, 160 nm, 180 nm, 200 nm, 220 nm, 240 nm, 260 nm, 280 nm, 300 nm, 320 nm, 340 nm, 360 nm, 380 nm, 400 nm, 420 nm, 440 nm, 460 nm, 480 nm, 500 nm, 520 nm, 540 nm, 560 nm, 580 nm, 600 nm, 620 nm, 640 nm, 660 nm, 680 nm, 700 nm, 720 nm, 740 nm, 760 nm, 780 nm, 800 nm, or more, or any number in between.

In some embodiments, the gold nanoparticle can have a layer structure with two planes and four sides. In some embodiments, the thickness of the nanoparticles can be shorter than the width and/or the length of the planes. In some embodiments, the width and length of the gold nanoparticles can be, for example, between about 100 nm to about 2,000 nm. In some embodiments, the width and length of the gold nanoparticles can be between about 100 nm to about 1,000 nm, for example. In illustrative embodiments, the width and length of the gold nanoparticles can be between about 10 nm to about 800 nm, e.g., 10 nm, 20 nm, 30 nm, 40 nm, 50 nm, 60 nm, 70 nm, 80 nm, 90 nm, 100 nm, 120 nm, 140 nm, 160 nm, 180 nm, 200 nm, 220 nm, 240 nm, 260 nm, 280 nm, 300 nm, 320 nm, 340 nm, 360 nm, 380 nm, 400 nm, 420 nm, 440 nm, 460 nm, 480 nm, 500 nm, 520 nm, 540 nm, 560 nm, 580 nm, 600 nm, 620 nm, 640 nm, 660 nm, 680 nm, 700 nm, 720 nm, 740 nm, 760 nm, 780 nm, 800 nm, or more, or any number in between. In some embodiments, the thickness of the gold nanoparticles can be between about 10 nm to about 200 nm. In some embodiments, the thickness of the gold nanoparticle can be between about 10 nm to about 80 nm, for example. In some embodiments, the thickness of the gold nanoparticle can be between about 10 nm to about 50 nm.

In some embodiments, the gold nanostructure can include one or more gold nanoparticles, one or more of which optionally can be different in shape, size, volume, and weight, for example. The number of gold nanoparticles included in a nanostructure can vary depending upon the size, shape, volume and weight of the gold nanoparticles. In some embodiments, one or more gold nanoparticles with various shapes, sizes, volumes, and weights can be included in the same gold nanostructure if desired.

Modification of certain conditions and components during the manufacturing of the gold nanostructure can influence a variety of physical, chemical, and functional properties of the gold nanostructure, and that such modifications are within the scope of the disclosure. Methods of producing a particular gold nanostructure can be selected according to a desired use of the nanostructure.

In various embodiments, one or more of the gold nanoparticles are deposited on or adhered to the dielectric material, which is coated onto part of or onto the entire substrate. Illustrative methods of adhering one or more gold nanoparticles onto the dielectric material include, but are not limited to, a chemical vapor deposition method, an atomic layer deposition method, electroless plating, and a molecular beam epitaxy method, which are well known in the field of technology.

In some embodiments, the gold nanoparticle can have any two-dimensional or three-dimensional structure. In general, when the gold nanoparticle is contacted with and adhered to the dielectric material, only part of the gold nanoparticle is contacted with the dielectric material. The part of the gold nanoparticle that is not contacted with the dielectric material is also generally not contacted with other gold nanoparticles. For example, when the gold nanoparticle has a layer structure having two planes and four sides, one of the two planes as well as the four sides of the gold nanoparticle may be in contact with the dielectric material, while the remaining plane is not in contact with, for example, the substrate, the dielectric material or any other gold nanoparticles. In some embodiments, each of the one or more gold nanoparticles assembled in the gold nanostructure may be separated from the others and at least partially surrounded by the dielectric material.

In some embodiments, gold nanostructures that include one or more gold nanoparticles, the dielectric material, and the substrate can be shaped in a variety of configurations, such as but not limited to, a layer or sheet, a cylinder, a sphere, a rod, a tubular structure, a fiber, any type of hexahedron, or any regular or irregular shaped two-dimensional or three dimensional structure. In addition, the size (e.g. diameter, width, length, and height) of the gold nanostructure can vary.

In some embodiments, the shape and the size of a gold nanostructure can be selected at least in part according to its desired function. For example, if the gold nanostructure is designed to be used in or transported through the vein of a human, where the average diameter of veins in a human body ranges between about 1 μm to about 1,000 μm, the gold nanostructure can be manufactured to fit within the vein. For such a purpose, a gold nanostructure that has a needle-like shape and a diameter of between about 1 μm to about 1,000 μm can be used, for example. If the gold nanostructure is designed to be used to treat a large group of cells e.g. a human face, the size (e.g. diameter, width, length, and height) of the gold nanostructure can be between about several centimeters to about several dozen centimeters depending on the size of the target cell area. In some embodiments, where desirable, the gold nanostructures can be manufactured to have a layer structure such that several target cells can be aligned on the surface of a face of one of the layers, allowing for the simultaneous treatment of target cells.

FIGS. 1A and 1B illustrate an example of a method of manufacturing a nanostructure. A planar substrate 10 is coated with a dielectric material 20. A gold nanopattern 25 is formed from two or more gold nanoparticles 30 on the dielectric material-coated substrate. The substrate 10 coated with the dielectric material 20 and the nanopattern 25 can then be rolled and sharpened to form a needle-like structure 35. The needle-like structure 35 can be connected to an optical fiber (not shown) through which light (hv) 40 can be transmitted. The needle-like structure 35 is configured to emit heat 60 as a result of transmission of the light 40. FIG. 1B depicts a magnified, cross-sectional view of the rolled-up structure of the needle-like gold nanostructure 50

A non-limiting illustrative method of manufacturing one embodiment of the gold nanostructure follows. The gold nanostructure can include a substrate, a dielectric material, and gold nanoparticles. The substrate is a square-shaped stainless steel foil with a 50.1 μm (width)×50.1 μm (length)×50 μm (thickness) size. The dielectric material is iron oxide. The gold nanoparticle is a square-shaped layer with a 400 nm (width)×400 nm (length)×30 nm (thickness) size.

Iron metal is coated on one plane of the square-shaped stainless steel substrate via the atomic layer deposition method, which is well known in the art. The thickness of iron coated onto the substrate is between about 50 nm to 150 nm. Once coated with iron, the stainless steel substrate is exposed to the air, which contains oxygen. This iron oxide coated substrate is kept in the air for 72 hours to completely oxidize the iron. Once the iron on the surface of the stainless steel is oxidized, it becomes iron oxide.

Gold nanoparticles are adhered to the iron oxide-coated stainless steel foil. One plane and four side lanes of the gold nanoparticles are contacted with iron oxide. A total 10,000 (100 horizontally and 100 vertically) gold nanoparticles are regularly placed on the iron oxide at an interval of 100 nm. The gold nanoparticles next to the edge of the iron oxide-coated substrate are positioned 100 nm from the edge of the substrate. Placing and adhering the 10,000 gold nanoparticles to the iron oxide-coated substrate in the regularly arrayed structure can be achieved via a chemical vapor deposition method, an atomic layer deposition method, an electroless plating, a molecular beam epitaxy method, or any other suitable technique. Among the two planes and four sides present in the gold nanoparticle, one plane and four sides are in contact with iron oxide while the other plane is not. Also, due to the 100 nm interval between adjacent gold nanoparticles, all gold nanoparticles are separated from each other.

The resulting gold nanostructure is a 50.1 μm (width)×50.1 μm (length)×50.08 to 50.18 μm (thickness) square-shaped layer. As the thickness of iron oxide is variable between 50 and 150 nm, the thickness of the gold nanostructure is variable accordingly. The gold nanostructure in this particular example has three layers; the bottom is the stainless steel substrate, the middle is iron oxide dielectric material, and the top is gold nanoparticles.

Depending on the need of a certain application, the gold nanostructure of the foregoing example with a square-shaped layer structure can be used without further modification. In some embodiments, a gold nanostructure with a square-shaped layer structure can be further shaped or processed. For example, a gold nanostructure with a square-shaped layer structure can be rolled and sharpened into a needle-like structure. In some embodiments, the gold nanoparticles of the needle-like structure can be externally situated, with the substrate in the interior, and the dielectric iron oxide present between gold nanoparticles and the substrate.

Several features of the SPR-induced heating can be varied depending on the size of the gold nanostructure, the purity of the materials in the gold nanostructure, and the shape of the gold nanostructure. As a result, the choice of components and manipulation thereof during manufacture of the gold nanostructure can be modified according to the needs of a particular application.

Generally, electromagnetic radiation provided to the gold nanostructure results in the emission of localized heat. In general, the electromagnetic radiation includes visible (Vis) to near infra red (IR) light (near IR). The near IR generally represents electromagnetic radiation that lies between the visible and microwave portions of the electromagnetic spectrum. The wavelength of near IR light used in certain embodiments disclosed herein can range from about 700 to about 2,500 nm. In some embodiments, the wavelength of near IR light used in the methods disclosed herein can range from about 800 to about 1600 nm, for example. In illustrative embodiments, the wavelength of near IR light can range for example, from about 800 to about 1200 nm.

In various embodiments, near-IR light can be provided to the gold nanostructure to induce local heating. In certain embodiments, near IR light can be provided or directed to the gold nanostructure through a radiation energy transmitting conduit which further can be connected to a radiation energy source. Alternatively near-IR can be provided or directed to the gold nanostructure from a radiation energy source that is not connected to the gold nanostructure.

Some embodiments disclosed herein relate to devices that are configured to provide near IR light to a gold nanostructure. In some embodiments, the devices can include, for example, a gold nanostructure and a radiation energy transmitting conduit, and optionally a radiation source connected to the conduit.

The SPR-induced heating technology linked to a transmitting conduit can be useful for various applications, including for example, applications and uses with animals, including humans. The gold nanostructures connected with or in combination with a physical structure such as a conduit can be used to specifically localize the gold nanostructure to a target in the body of an animal, to provide heat to the target efficiently and precisely, and to remove the gold nanostructure easily and completely from the body after treatment. For example, a physician or other operator can use the gold nanostructures as described herein, including those with an energy transmission conduit, to safely and effectively apply heat to specific locations.

In some embodiments, the radiation energy transmitting conduit can have, for example, two ends, one that is at proximal end and another at a distal end. The proximal end typically can be connected to or configured to receive energy from the radiation energy source, and the distal end typically can be connected to the gold nanostructure. The gold nanostructure and the radiation energy source can be connected to or in contact with the conduit permanently or temporarily.

In some embodiments, the gold nanostructures can be shaped to be easily connected with the conduit during the manufacturing. In some aspects, more than one of the gold nanostructures can be connected with a conduit and used simultaneously

The radiation energy transmitting conduit in various embodiments generally can be capable of transmitting electromagnetic radiation energy, such as but not limited to near IR light. In some embodiments, the radiation energy transmitting conduit can be configured to transmit near IR light with a wavelength ranging between about 700 to about 2,500 nm, for example. In some embodiments the radiation energy transmitting conduit can be configured to transmit near IR light with a wavelength ranging between about 800 to about 1,600 nm, for example. In some illustrative embodiments, the radiation energy transmitting conduit is configured to transmit near IR light with a wavelength ranging between about 800 to about 1,200 nm.

The radiation energy transmitting conduit can be made of any material that capable of transmitting electromagnetic radiation energy such as near IR light. In some embodiments, the radiation energy transmitting conduit can be, for example, an electric wire that can transmit electromagnetic radiation energy such as near IR light. In some embodiments, the radiation transmitting conduit can be, for example, a tube with the outer walls surrounded by an energy insulating material. In general, near IR energy provided by the radiation energy source is transmitted though the tube and reaches the gold nanostructure that is connected to the distal end of the conduit.

In some embodiments, the conduit can include an optical fiber, through which near IR provided from the radiation energy source can be transferred. Accordingly, in some embodiments, the optical fiber can include a fiber configured to transmit electromagnetic energy radiation such as near IR along its length. Optical fibers useful in the embodiments disclosed herein can be composed of a variety of materials, including but not limited to glass, plastic and/or other chemicals, for example. Near IR light transmitted through such a conduit is provided to the gold nanostructure resulting in heat emission around the gold nanostructure.

In some embodiments an optical fiber can be connected with a needle-shaped gold nanostructure. The needle-shaped nanostructure can be configured to be nanometer-scale to micrometer-scale, in order to accommodate the diameter of the optical fiber, which can have a diameter, for example, of about several hundred micrometers. Such optical fiber/nanostructure devices can be used in any of the applications described herein, for example.

In some embodiments, the radiation transmitting conduit can be elongated. The length of the conduit can be extended to be long enough to allow the gold nanostructure to reach to the desired area that is to be heated. For example, the conduit can have a length that permits the gold nanostructure to reach an area inside the body of a mammal, including a human, while the conduit can still transmit energy to the nanostructure.

In some embodiments, a radiation transmitting conduit can be connected to both a gold nanostructure and to a controlling device. For example, in embodiments where the gold nanostructure is administered to a human (or an animal or mammal) and heats a cellular or non-cellular tissue therein, the controlling device can help a doctor or an operator (or a veterinarian) to control various aspects of the procedure. For example, the controlling device can be configured to assist with the administration and localization of the gold nanostructure, the induction of heat, the removal of the gold nanostructure from the body, and any other aspects of the procedure. As used herein, the terms “doctor” and “operator” can refer to any person(s) who utilizes the gold nanostructure for any purpose.

In some embodiments, a visual aid, such as but not limited to, an endoscope can be used to facilitate the delivery of a gold nanostructure to animals, including humans and other mammals, for example. An endoscope provides the ability to visualize the interior of the animal, and in part can provide direct or indirect visual inspection of the area of interest. Examples of endoscopic procedures include, but are not limited to, an esophagogastroduodenoscopy, a gastroscopy, a colonoscopy, a proctosigmoidoscopy, an endoscopic retrograde cholangiopancreatography, a rhinoscopy, a bronchoscopy, a cystoscopy, a colposcopy, a falloscopy, a laparoscopy, an arthroscopy, a thoracoscopy, a mediastinoscopy, a panendoscopy, and an angioscopy.

In illustrative embodiments, a gold nanostructure connected to a radiation transmitting conduit can be administered to mammals thorough a natural opening or through a minimal incision on the desired body part. The gold nanostructure can be localized internally using, for example, an endoscopic procedure. Electromagnetic energy such as near IR can be provided to the localized gold nanostructure from the radiation energy source through the conduit.

In some embodiments, the gold nanostructure and/or the conduit can be attached to a viewing system such as an endoscope and localized to the area to be heated using the viewing system.

In some embodiments, if the area desired to be heated is present on a surface of an animal, such as a human or non-human mammal, the gold nanostructure can be localized to the target area with or without help of another type of viewing system such as a magnifier.

In some embodiments, the gold nanostructure can be coupled with an imaging system. Such an imaging system optionally can include, but is not limited to, a CT scanning system, a magnetic resonance imaging (MRI) system, and/or a nuclear magnetic resonance (NMR) system, for example. The imaging system can facilitate the monitoring of the thermal treatment given by the gold nanostructure a doctor or an operator, for example.

The components of the gold nanostructure, including the one or more gold nanoparticles, the dielectric material, and the substrate, are not biodegradable. Therefore, in some aspects the gold nanostructure can remain in the animal (e.g., a non-human mammal or a human for an extended period of time. The gold nanostructure can be removed and some embodiments of the present disclosure provide efficient ways to remove the gold nanostructure from the targeted area or the body after heat induction. For example, the gold nanostructure can be removed from the body when the radiation energy transmitting conduit or the endoscope, is removed, for example.

Some embodiments herein provide methods of treating various targets, including cellular and non-cellular tissues with heat, using the gold nanostructures disclosed herein. As SPR-induced heating is generally very safe, this thermal treatment by the gold nanostructure can be used for cellular and non-cellular targets present in living organisms including humans.

In some embodiments, the gold nanostructure can be used to remove or destroy cellular tissues. In illustrative embodiments, the cellular tissues can be, for example, cancerous cells or tissues. In general, cancer cells aberrantly overgrow compared to the normal cells, and such cells can grow in many different parts of a human body. Some non-limiting examples of cancerous cells or tissues that can be treated with the nanostructures and devices disclosed herein include, but are not limited to, cancerous tissues of the colon, stomach, lungs, throat, uterus, bladder, and esophagus. These organs can be relatively easily tested via an endoscopic observation for the existence of hyper-proliferating cells or tissues, which can further develop into cancer. In some embodiments, an endoscopic system and gold nanostructure can be administered to the desired organ simultaneously. If the cancerous tissues or cells are found during the endoscopic investigation, a doctor or an operator can destroy or remove such cancerous tissues immediately by inducing local heat generated by the gold nanostructure. Furthermore, an imaging system, such as but not limited to, a CT scanning system, an NMR system, and an MRI system can be used along with the gold nanostructure to monitor the removal or destruction of the targeted cancerous or non-cancerous cells or tissues after heat treatment if desired.

In some embodiments, the nanostructures and devices disclosed herein can be used for the removal or destruction of non-cellular tissues, for example. As one example, cholesterol is a lipid that plays an essential role in animal biology, including forming part of cell membranes. While cholesterol is essential, abnormally high cholesterol levels have been implicated in various conditions including cardiovascular disease. Excessive cholesterol can accumulate in arteries and cause atheromas, a deposit or degenerative accumulation of lipid-containing plaques on the innermost layer of the wall of an artery. This condition can further lead to various types of heart attack, stroke, and peripheral vascular disease.

In some embodiments, SPR-induced hyperthermia can be used to treat atheroma, including atheroma caused by or contributed to by cholesterol. If removal of plaque, including but not limited to accumulated cholesterol, is desired, one can administer gold nanostructures along with an angioscope via a minimal incision. Once the gold nanostructures are localized to the plaque, hyperthermia can be induced to remove the cholesterol, and optionally, other obstructing materials from the artery.

In some embodiments, as severity of the plaque may be monitored by the accompanying angioscope, the doctor or the operator can manipulate the time of the light transmission provided to the gold nanostructure to provide the proper strength of hyperthermia to remove the plaque without damaging the artery or vein. For example, the operator or doctor can visual monitor the removal of the plaque and/or the effect of the localized heating. Based upon what is observed the operator can continue to induce heating of the plaque or other material, can discontinue the heating, or can start and stop, repeatedly, for example, as needed. In general, the gold nanostructure that is connected with a radiation energy transmitting conduit and a radiation energy source can be administered to a vein using an angioscope to monitor and assist in placement of the nanostructure. In some examples, the doctor or the operator can locate the gold nanostructure at the edge of the plaque and induce heat if plaque(s) occupies some area of the vein well. As the area of plaque is smaller after heat induction and such change is monitored by the angioscope, the doctor or the operator can move the gold nanostructure close to the remaining plaque. When the targeted plaque is substantially or completely removed by induced heat and such change is monitored by the angioscope, the doctor or the operator can stop transmitting light to the gold nanostructure by controlling the radiation energy source. Depending on the size of plaque, the doctor or the operator also can shorten or extend the time of light transmission to the gold nanostructure. In addition, if the plaque is in close proximity to the vein well and such short distance is monitored by the angioscope, the doctor or the operator can provide short, repeated applications of heat induction to minimize the unwanted damage to the vein.

In some embodiments, the thermal treatment induced by the gold nanostructures can be used for a cosmetic purpose. For instance, age spots, which are in general the accumulation of the pigment (e.g., caused by exposure to the sun), often occur on the skin. Age spots can be treated or reduced using gold nanostructure-induced heating. Similarly acne flare ups may be reduced by treatment via hyperthermia using the gold nanostructures.

In some embodiments, various skin disorders caused by infections of bacteria, fungi, virus and other microorganisms can be treated by heating such microorganisms and infected cells with the gold nanostructures described herein.

In certain applications, the gold nanostructure-induced thermal treatment can be used to remove excessive hair, for example. Hairs grow from hair cells that exist under the skin. The gold nanostructure can be directly inserted into the hair follicle in order to damage the hair cells to reduce further hair growth.

In some embodiments, the area desired to be heated can be non-living. For instance, the gold nanostructure can be used to increase the temperature of any liquid such as, but not limited to water and oil. Alternatively the gold nanostructure can be used to melt certain types of solids, such as but not limited to ice and fat. This gold nanostructure can be used in a variety of areas and subjects desired to be heated within the scope of the disclosure. Therefore, this disclosure should be considered to include any potential target area or subject desired to be heated and can be treated with the gold nanostructure, wherein the potential target area or subject can be living or non-living.

What is described in this specification can be modified in a variety of ways while remaining within the scope of the claims. Therefore all embodiments disclosed herein should be considered as illustrative embodiments of the present disclosure and should not be considered to represent the entire scope of the disclosure.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A gold nanostructure, comprising:
a substrate;
a dielectric material coated on the substrate; and
one or more gold nanoparticles adhered to the dielectric material-coated substrate.

2. The gold nanostructure of claim 1, wherein said gold nanoparticles are from about 100 to 2,000 nm wide and from about 10 to 200 nm thick.

3. The gold nanostructure of claim 1, wherein said dielectric material is selected from the group consisting of iron oxide, aluminum oxide, titanium oxide and combinations thereof.

4. The gold nanostructure of claim 1, wherein said substrate is stainless steel.

5. A device comprising:
a gold nanostructure, wherein the gold nanostructure comprises a substrate, a dielectric material coated on the substrate, and one or more gold nanoparticles adhered to the dielectric material-coated substrate; and
a radiation energy transmitting conduit, wherein the radiation energy conduit is configured to connect to the gold nanostructure.

6. The device of claim 5, wherein said electromagnetic radiation energy transmitting conduit comprises an optical fiber.

7. The device of claim 5, wherein said radiation energy transmitting conduit is connected to a radiation energy source.

8. The device of claim 7, wherein said radiation energy source provides radiation of wavelengths ranging from about 800 to about 1,200 nanometers through said conduit to said gold nanostructure.

9. The device of claim 5, further comprising an imaging system to monitor the condition of a heated cellular or non-cellular tissue by the gold nanostructure, wherein said imaging system is selected from the group consisting of a CT scanning, a NMR, a MRI, and combinations thereof

10. A method of reducing or destroying a cellular or non-cellular tissue comprising:
localizing a gold nanostructure to the cellular or non-cellular tissue, wherein a gold nanostructure comprises a substrate, a dielectric material coated on the substrate, and one or more of gold nanoparticles adhered to the dielectric material-coated substrate; and
providing radiation to the gold nanostructure to induce heat.

11. The method of claim 10, wherein said cellular tissue is a cancerous cellular tissue.

12. The method of claim 10, wherein said cellular tissue is a non-cancerous cellular tissue.

13. The method of claim 10, wherein said cellular or non-cellular tissue is present in a mammal.

14. The method of claim 10, wherein said radiation is of wavelengths from about 800 nanometers to about 1,200 nanometers.

15. The method of claim 10, wherein said localizing the gold nanostructure to the cellular or non-cellular tissue is conducted with an endoscopic system.

16. The method of claim 15, wherein said endoscopic system is selected from the group consisting of an esophagogastroduodenoscopy, a gastroscopy, a colonoscopy, a proctosigmoidoscopy, an endoscopic retrograde cholangiopancreatography, a rhinoscopy, a bronchoscopy, a cystoscopy, a colposcopy, a falloscopy, a laparoscopy, an arthroscopy, a thoracoscopy, a mediastinoscopy, a panendoscopy, an angioscopy, and combinations thereof

17. The method of claim 13, further comprising removing the gold nanostructure from the mammal.

18. The method of claim 10, further comprising using an imaging system to monitor the condition of a heated cellular or non-cellular tissue by the gold nanostructure, wherein said imaging system is selected from the group consisting of a CT scanning, a NMR, a MRI and combinations thereof

19. A method of making a gold nanostructure, comprising:
coating a substrate with a dielectric material; and
adhering one or more gold nanoparticles to the dielectric material-coated substrate.

20. The method of claim 19, further comprising:
forming said gold nanostructure into a shape, wherein said shape is capable of being attached to a radiation transmitting conduit.

21. The method of 20, further comprising:
rolling and sharpening the gold nanostructure such that it is configured to be a thin structure capable of being attached to a radiation transmitting conduit.

22. A method of making a device comprising:
connecting one or more gold nanostructures to a radiation energy transmitting conduit, wherein the one or more gold nanostructures include a substrate, a dielectric material coated on the substrate, and one or more of gold nanoparticles adhered to the dielectric material-coated substrate.

23. The method of claim 22, wherein said radiation energy transmitting conduit includes an optical fiber.

24. The method of claim 22, further comprising:
connecting the radiation energy transmitting conduit to a radiation energy source.

25. The method of claim 24, wherein said radiation energy source provides radiation of wavelengths ranging from about 800 to about 1,200 nanometers through said conduit to said gold nanostructure.

26. A method of generating heat at a desired location, comprising:
localizing a gold nanostructure at the desired location, wherein the gold nanostructure comprises a substrate, a dielectric material coated on the substrate, and one or more gold nanoparticles adhered to the dielectric material-coated substrate; and
providing radiation to the gold nanostructure to induce heat at the desired location.

27. The method of claim 26, wherein said desired location is a cellular tissue.

28. The method of claim 26, wherein the desired location is non-living.
